(19) United States
(12) Patent Application Publication
(10) Pub. No.: US 2007/0061002 A1
(43) Pub. Date: Mar. 15, 2007

(54) Attachment of material to an implantable frame by cross-linking

(75) Inventors: Ram H. Paul JR., Bloomington, IN (US); Brian C. Case, Lake Villa, IL (US); Grant T. Hoffman, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/514,443

(22) Filed: Sep. 1, 2006

Related U.S. Application Data
(60) Provisional application No. 60/713,453, filed on Sep. 1, 2005.

(51) Int. Cl. A61F 2/90; A61L 33/00; B05D 3/00; A61F 2/06

(52) U.S. Cl. 623/1.13; 427/2.1; 623/1.24; 623/1.16

(57) ABSTRACT

Medical devices for implantation in a body vessel, and methods of using and making the same, are provided. A medical device includes a frame and a cross-linkable material having at least one cross-linked region and a non cross-linked region, where the cross-linked region maintains the cross-linkable material in connection to the frame. A method of making an implantable medical device includes providing a frame and covering the frame with a cross-linkable material. The method also includes cross-linking at least one region of the cross-linkable material by joining a first region of the cross-linkable material to a second region of the cross-linkable material to form a point of attachment. A method of treating a subject includes the step of implanting the medical device at a point of treatment.

### RELATED APPLICATIONS

This application claims priority to provisional application No. 60/713,453 filed on Sep. 1, 2005, the entire disclosure of which is incorporated by reference herein.

### TECHNICAL FIELD

The present invention relates to medical devices. More particularly, the invention relates to medical devices for implantation in a body vessel comprising a material attached to a frame.

### BACKGROUND

Many vessels in animals transport fluids from one body location to another. Frequently, fluid flows in a substantially unidirectional manner along the length of the vessel. For example, veins in the body transport blood to the heart and arteries carry blood away from the heart.

Recently, various implantable medical devices and minimally invasive methods for implantation of these devices have been developed to deliver these medical devices within the lumen of a body vessel. These devices are advantageously inserted intravascularly, for example from an implantation catheter. For example, implantable medical devices can function as a replacement venous valve, or restore native venous valve function by bringing incompetent valve leaflets into closer proximity. Such devices can comprise an expandable frame configured for implantation in the lumen of a body vessel, such as a vein. Venous valve devices can further comprise features that provide a valve function, such as opposable leaflets.

Many devices, such as implantable medical devices, comprise a material attached to a frame. The inclusion of a material to a medical device often requires a connection between the frame and material. The dynamic environment in which the medical device is placed requires a connection that maintains the material in a desired structured orientation with respect to the frame.

There exists a need in the art for an implantable medical device that comprises a material suitably attached to a frame. Materials attached to an implantable frame without use of adhesives are particularly desirable. For example, a portion of material can be joined to an implantable frame by treating the material and/or frame with conditions that promote the formation of cross-link chemical bonds that suitably secure portions of the material to a frame.

### SUMMARY

The invention relates to medical devices for implantation in a body vessel. More specifically, preferred embodiments relate to a medical device comprising a frame with a cross-linkable material attached to the frame by formation of cross-link chemical bonds to the material. The cross-linkable material can be attached to itself or to the frame with agents such as tissue adhesives, cross-linkers, or natural materials such as fibrin. The cross-linkable material also can define structural components that facilitate attachment to a frame.

In one aspect of the present invention, a medical device for implantation in a body vessel comprises a frame and a cross-linkable material having at least one cross-linked region and a non cross-linked region, where the cross-linked region maintains the cross-linkable material in connection to the frame.

The cross-linked region can comprise cross-linkable material attached to the frame or cross-linkable material attached to itself. The cross-linked region can allow cross-linkable material to move relative to the frame to any suitable extent or any suitable direction. The cross-linked region also can allow cross-linkable material to remain substantially fixed with respect to the frame. Cross-linkable material can include graft material or valve leaflet. Preferably, the cross-linkable material is a flexible material.

In another aspect of the present invention, a method of making an implantable medical device comprises providing a frame and covering the frame with a cross-linkable material. The method further comprises cross-linking at least one region of the cross-linkable material, wherein the cross-linking comprises forming a cross-linked region joining a first region of the cross-linkable material with a second region of the cross-linkable material.

In yet another aspect of the present invention, a method of treating a subject comprises implanting a medical device at a point of treatment, wherein the medical device comprises a frame and a cross-linkable material having a cross-linked region where the cross-linked region maintains the cross-linkable material in connection to the frame.

The invention includes other embodiments within the scope of the claims, and variations of all embodiments, and is limited only by the claims made by the Applicants. Additional understanding of the invention can be obtained by referencing the detailed description of embodiments of the invention, below, and the appended drawings.

### BRIEF DESCRIPTION OF THE DRAWINGS

FIGS. 1A and 1B is a partially sectioned side view of a medical device according to an embodiment of the present invention;

FIG. 2 is an enlarged cross-sectional view of the cross-linked region taken along line A-A′.

FIG. 3 is a partially sectioned side view of a medical device according to an embodiment of the present invention.

FIG. 4 is an enlarged cross-sectional view of the cross-linked region taken along line B-B′.

FIG. 5 is a side view of a valve according to an embodiment of the present invention having cross-linkable material attached to a frame.

FIG. 6 is a left side view of the valve depicted in FIG. 5.

FIG. 7 is a side view of a valve according to an embodiment of the present invention.

FIG. 8 is an enlarged cross-sectional view of the cross-linked region taken along line C-C′.

FIG. 9 is an enlarged cross-sectional view of the cross-linked region taken along line C-C′.

FIG. 10 is an enlarged sectional view of the cross-linked region taken along line D-D′.

FIG. 11 is a partially sectioned side view of a medical device according to an embodiment of the present invention.

FIG. 12 is a perspective view of a medical device according to an embodiment of the present invention.

FIG. 13 is an enlarged cross-sectional view of the cross-linked region taken along line E-E′.

FIG. 14 is a perspective view of a medical device according to an embodiment of the present invention.

FIG. 15 is a perspective view of a medical device according to an embodiment of the present invention in a closed configuration.

FIG. 16 is a perspective view of a medical device according to an embodiment of the present invention in an open configuration.

### DETAILED DESCRIPTION OF EMBODIMENTS

The following detailed description and appended drawings describe and illustrate various exemplary embodiments of the invention. The description and drawings serve to enable one skilled in the art to make and use the invention, and are not intended to limit the scope of the invention in any manner.

The invention provides medical devices for implantation in a body vessel, methods of making the medical devices, and methods of treatment that utilize the medical devices.

As used herein the terms “comprise(s),” “include(s),” “having,” “has,” “contain(s),” and variants thereof, are intended to be open-ended transitional phrases, terms, or words that do not preclude the possibility of additional acts or structure.

As used herein, the term “body vessel” means any tube-shaped body passage lumen that conducts fluid, including but not limited to blood vessels such as those of the human vasculature system, biliary ducts and ureteral passages.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

As used herein, “endolumenally,” “intraluminally” or “transluminal” all refer synonymously to implantation placement by procedures wherein the prosthesis is advanced within and through the lumen of a body vessel from a remote location to a target site within the body vessel. In vascular procedures, a medical device will typically be introduced “endovascularly” using a catheter over a guidewire under fluoroscopic guidance. The catheters and guidewires may be introduced through conventional access sites to the vascular system, such as through the femoral artery, or brachial and subclavian arteries, for access to the coronary arteries.

A “biocompatible” material is a material that is compatible with living tissue or a living system by not being toxic or injurious and not causing immunological rejection.

The invention relates to medical devices for implantation in a body vessel. More specifically, embodiments of the invention relate to a medical device comprising a cross-linkable material maintained in connection to a frame by one or more points of attachment.

The term “cross-link” refers to the formation of a chemical bond between two polymers or molecules by formation of one or more covalent bonds between the two polymers or molecules. Preferably, a medical device comprises a flexible material comprising a polymer capable of forming cross-link bonds to itself or to another polymer or molecule.

The term “frame” refers to any biocompatible frame suitable for implantation within a body vessel. Preferably, a frame can expand from a compressed, or unexpanded, delivery configuration to one or more radially expanded deployment configurations, for example through self-expansion or balloon expansion of the frame. The expanded configuration can have any suitable cross-sectional configuration, including circular or elliptical. In one embodiment, the frame can be oriented along the longitudinal axis of a body vessel in the expanded or compressed configurations.

As used herein, a “cross-linkable material” refers to any material suitable for attachment about a frame of a medical device. Preferably, the cross-linkable material maintains flexibility to move in response to fluid contacting the material in a body vessel.

As used herein, a “point of attachment” refers to a location wherein a bond is formed thereby linking two adjacent surfaces.

As used herein, a “cross-linked region” refers to a region comprising a first region and second region that are bonded together via cross-linking.

Cross-Linked Regions and Points of Attachment

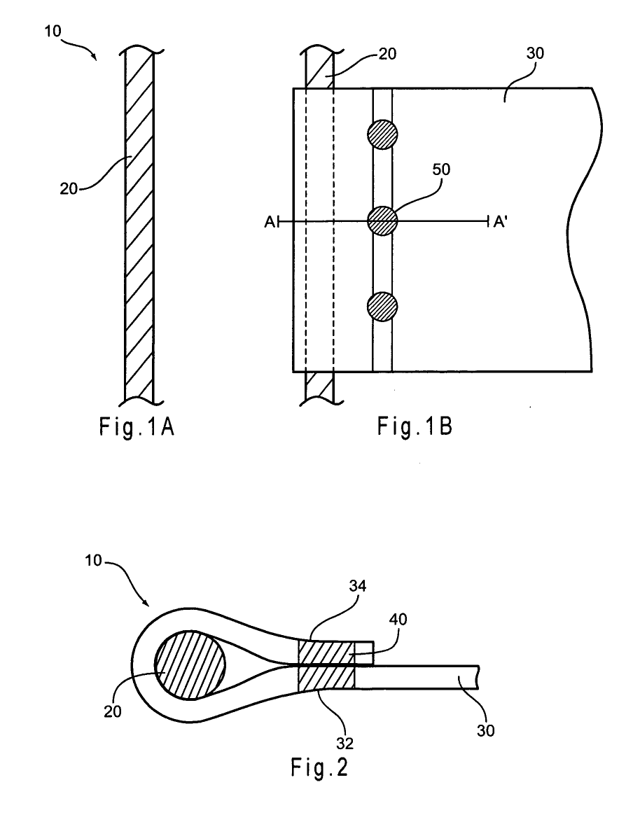

Referring to FIG. 1A and FIG. 1B, a medical device 10 is provided for implantation within a vessel of a body lumen. The medical device 10 of FIG. 1B illustrates a partially sectioned side view of a medical device according to an embodiment of the present invention. FIG. 2 illustrates an enlarged cross-sectional view of the cross-linked region taken along line A-A′ of FIG. 1B which includes the cross-linked region 40 forming a portion of point of attachment 50, and as well as a portion of cross-linkable material 30 and a portion of frame 20. The medical device 10 includes a frame 20 and a cross-linkable material 30 having at least one cross-linked region 40 and a non cross-linked region, where the cross-linked region 40 maintains the cross-linkable material 30 in connection to the frame 20 (see FIG. 2). Cross-linkable material 30 is attached to frame 20 by one or more points of attachment 50. Points of attachment 50 are formed by one or more cross-linked regions 40 of the cross-linkable material 30. The cross-linkable material 30 can be cross-linked to itself or to a frame 20 to form a point of attachment 50. The point of attachment 50 can be moveable relative to the frame 20.

FIG. 1 illustrates a suitable frame 20 for use in a medical device 10 according to an embodiment of the present invention. Referring to FIG. 2, the cross-linkable material 30 is attached to itself to maintain the connection to the frame 20. The cross-linkable material 30 preferably is suitable for use as a prosthetic valve leaflet. The cross-linkable material 30 is attached to itself by cross-linking a first region 32 of the cross-linkable material 30 to a second region 34 of the cross-linkable material 30. A point of attachment 50 is formed along a portion of the cross-linkable material 30 wherein the first region 32 of the cross-linkable material 30 is bonded to the second region 34 of the cross-linkable material 30.

FIG. 2 illustrates a cross linked region 40 including cross-linkable bonds between the first region 32 of the cross-linkable material 30 and the second region 34 of the cross-linkable material 30. The frame 20 is positioned between the first region 32 of the cross-linkable material 30 and second region 34 of the cross-linkable material 30, wherein the cross-linkable material 30 remains movable about the frame 20. The cross-linkable material 30 is overlapped over the frame 20 which allows the cross-linkable material 30 to remain movable with respect to the frame 20.

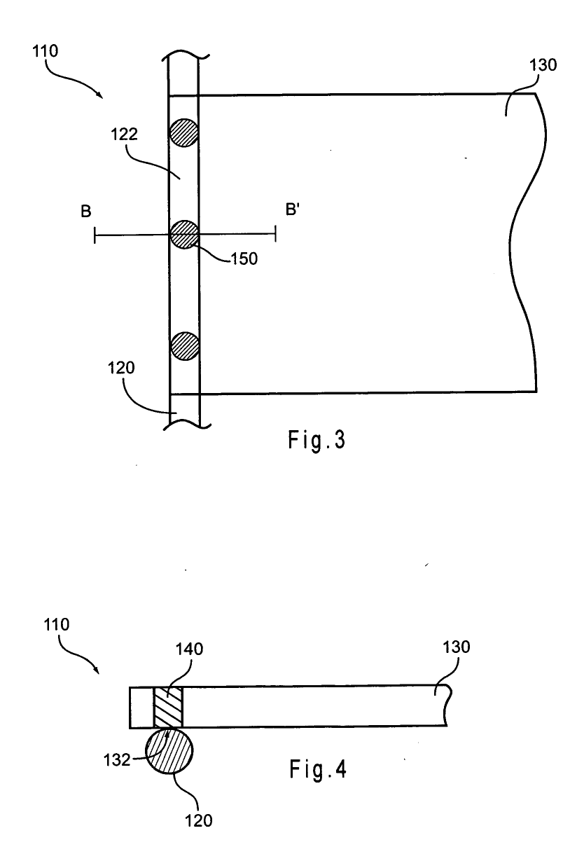

FIG. 3 illustrates a medical device 110 according to a second embodiment of the present invention. FIG. 4 is an enlarged cross-sectional view of FIG. 3 taken along line B-B′. In this embodiment, a cross-linkable material 130 is attached to a frame 120 to maintain a connection to the frame 120. The cross-linkable material 130 is attached to the frame 120 by cross-linking a first region 132 of the cross-linkable material 130 to a surface 122 of the frame 120. A point of attachment 150 is formed along a portion of the frame 120 wherein the first region 132 of cross-linkable material 130 is bonded to the surface 122 of the frame 120 (see FIG. 3). To improve the bond between the frame 120 and the cross-linkable material 130 various surface modification techniques can be utilized, such as grit-blasting, tumbling, chemical etching, plasma treatment, and any other suitable technique to prep surface for coating the medical device 10.

FIG. 4 illustrates a cross-linked region 140 including a cross-linkable bond between the first region 132 of cross-linkable material 130 and the surface 122 of the frame 120. The frame 120 is positioned against the first region 132 of cross-linkable material 130, wherein the cross-linkable bond connects the cross-linkable material 130 directly to the frame 120. The cross-linkable material 130 remains stationary with respect to the frame 120.

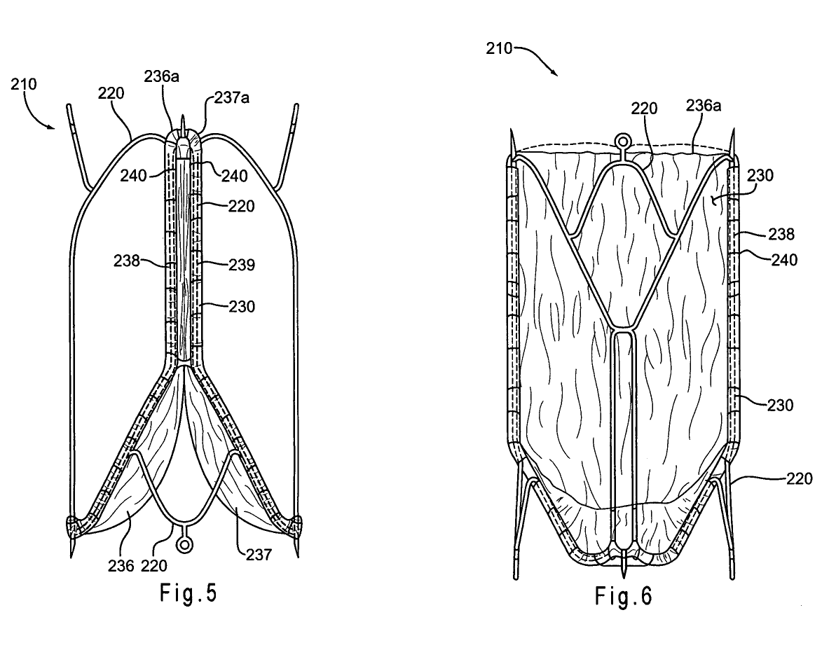

With reference to FIGS. 5-6, various side views of a valve 210 are illustrated according to an embodiment of the invention having cross-linkable material 230 secured to a frame 220. The medical valve 210 further provides two leaflets 236 and 237 in a first configuration for implantation in a patient. In particular, FIG. 5 provides a side view of a valve 210 viewed in a direction parallel to the coapting upper edges 236a and 237a of leaflets 236 and 237. FIG. 6 provides a view of the valve 210 depicted in FIG. 5 taken from one end.

As can be seen from FIGS. 5-6, leaflets 236 and 237 include respective free edges 236a and 237a for coaptation with one another and respective fixed edges 238 and 239 the central portion of which can be displaced toward the wall of a vascular vessel upon implantation of valve 210 in a body vessel with fluid flowing therethrough.

The cross-linkable material 230 comprising the edge of the leaflets 236, 237 is cross-linked to the frame 220 forming a plurality of cross-linking regions 240 along a portion of the cross-linkable material 230 attached to the frame 220.

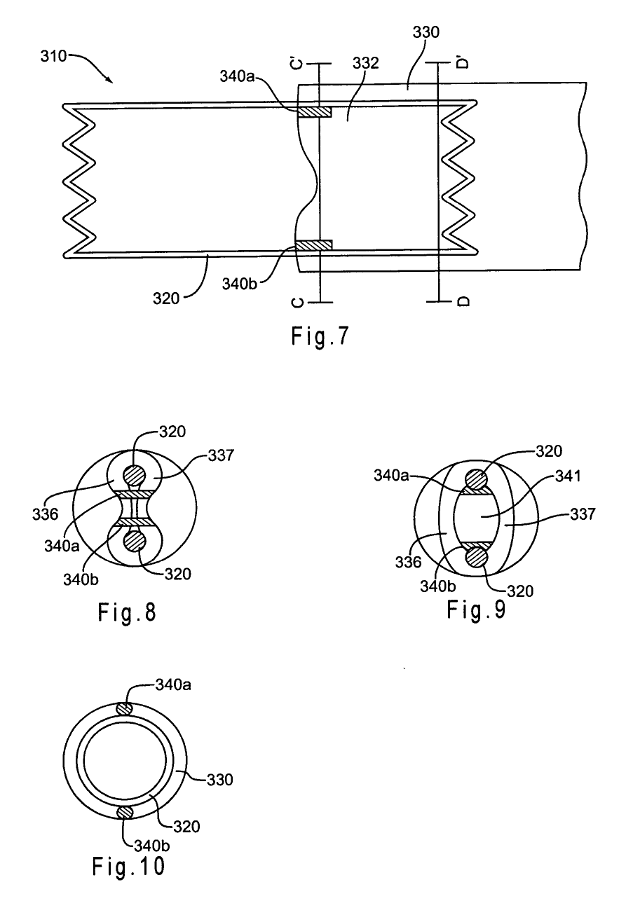

FIG. 7 illustrates an alternate valve 310 according to another embodiment of the invention. In this embodiment, a cross-linkable material 330 is attached to a frame 320 to maintain a connection to the frame 320. The cross-linkable material 330 extends along approximately half of frame 320. The valve 310 includes a first cross-linked region 340a and second cross-linked region 340b, wherein a first region 332 of the cross-linkable material 330 is cross-linked to a surface of the frame 320.

FIGS. 8-9 illustrate a cross-sectional view of the valve shown in FIG. 7 taken along line C-C′, wherein the cross-linkable material 330 defines leaflets 336, 337. FIG. 10 illustrates a sectional view of the cross-linked region taken along line D-D′. The leaflets are movable between a first and second position when the valve 310 is placed in a body lumen. In the first position, illustrated in FIG. 8, the leaflets 336, 337 permit fluid flow in a first direction. The pressure created by the flow of fluid exerts a force on one face of the leaflets 336, 337, displacing the leaflets 336, 337 toward a closed position. In the second position, illustrated in FIG. 9, the leaflets 336, 337 substantially prevent fluid flow in a second, opposite direction. When a pressure change and/or flow reversal occurs, the pressure created by the flow of fluid exerts a force on an opposite face of the leaflets 336, 337 displacing the leaflets 336, 337 toward an open position. By moving between these two positions, the leaflets 336, 337 provide a valving function to the valve 310, allowing it to regulate fluid flow. The leaflets 336, 337 further define a valve opening 341. The leaflets 336, 337 are movable between the first and second positions. While in the first position the leaflets 336, 337 come together to close the opening 341. In the second position, the opening 341 allows blood flow through the valve.

The dimensions and configurations of the cross-linkable material 330 can be optimized based upon the vessel in which the valve 310 is placed. The size and configuration selected will depend on several factors, including the vessel size, typical flow volumes and rates, and other factors depending on the configuration of the valve 310.

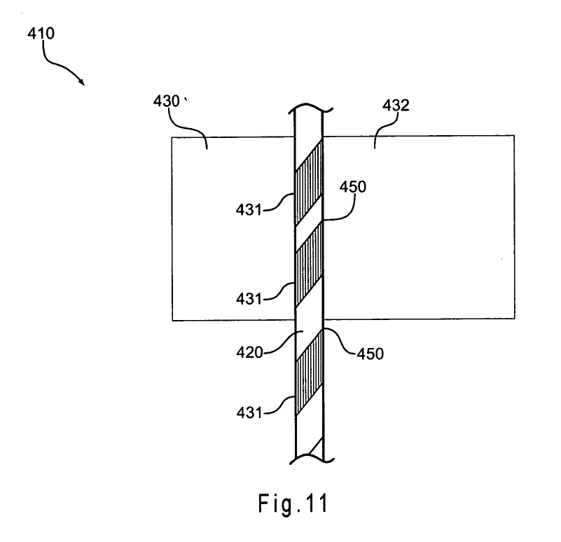

FIG. 11 illustrates a medical device 410 according to another embodiment of the present invention. In this embodiment, the cross-linkable material 430 is attached to a frame 420 to maintain a connection to the frame 420. The cross-linkable material 430 is attached to the frame 420 by cross-linking a first region 432 of cross-linkable material 430 to a surface of the frame 420. In addition, the device 410 can include a plurality of cross-linkable material 431 cross-linked along the surface of the frame 420. The sections of cross-linkable material 431 along the frame 420 can further provide points of attachment 450 along a portion of the frame 420 wherein the cross-linkable material 431 is bonded to the surface of the frame 420. The cross-linkable material 431 remains stationary with respect to the frame 420. The cross-linkable material 431 can include a combination of tissue and polymers depending on the particular design and configuration. For example, the cross-linkable material 431 located on the frame 420 can include an extra cellular matrix material, such as small intestine submucosa (SIS), while the remaining cross-linkable material 430 can comprise a tissue. The frame 420 can comprise one or more suitable polymers using conventional manufacturing techniques, such as braiding, coiling, or knitting.

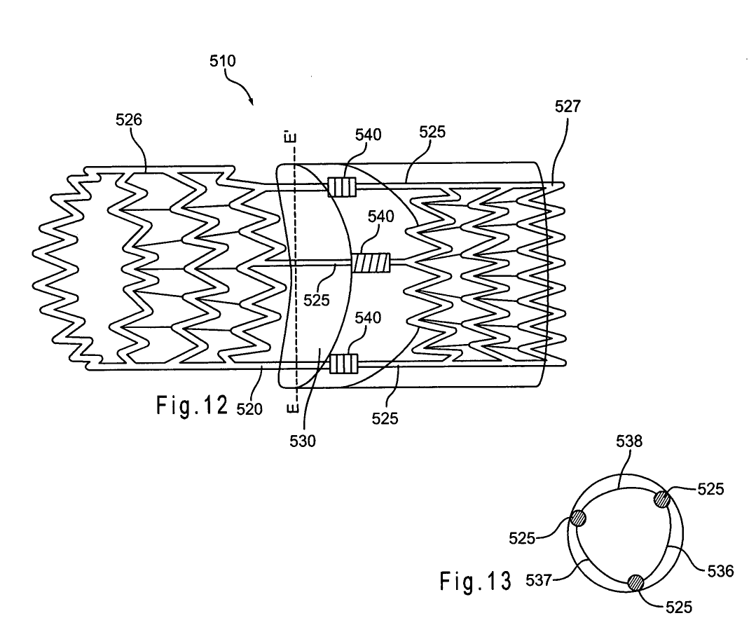

FIG. 12 illustrates a medical device 510 according to another embodiment of the present invention. The medical device 510 comprises a frame 520 having a cross-linkable material 530 cross-linked to the frame 520. The structure of the frame 520 consists of three bar members 525. In the embodiment, the cross-linkable material 530 is cross-linked to the bar members 525 of the frame 520. The frame 520 further includes a proximal portion 526 and a distal portion 527 connected to the bar members 525 of the frame 520. The proximal portion 526 and distal portion 527 of the frame 520 can include a Z or S shaped configuration, however, the configuration should not be construed as limiting the scope of invention. One of ordinary skill in the art would understand that other geometries may be used.

The cross-linkable material 530 can be attached to itself or the frame 520 at a location between the proximal portion 526 and distal portion 527 of the medical device 510. The cross-linkable material 530 is preferably located at the approximate longitudinal midpoint of the bar members 525 and extend to an end of the device 510 (see FIG. 12). In an alternate embodiment, the cross-linkable material 530 can cover the entire length of the medical device 510. The cross-linkable material 530 can further define a plurality of leaflets 536, 537, 538 wherein each leaflet 536, 537, 538 is cross-linked to a separate bar member 525. FIG. 13 illustrates a cross-sectional view of the device 510 shown in FIG. 12, wherein the number of leaflets 536, 537, 538 is equivalent to the number of bar members 525. In the illustration, the device 510 includes three leaflets and three bar members, however, in an alternate embodiment the number of leaflets and/or bar members may be larger or smaller.

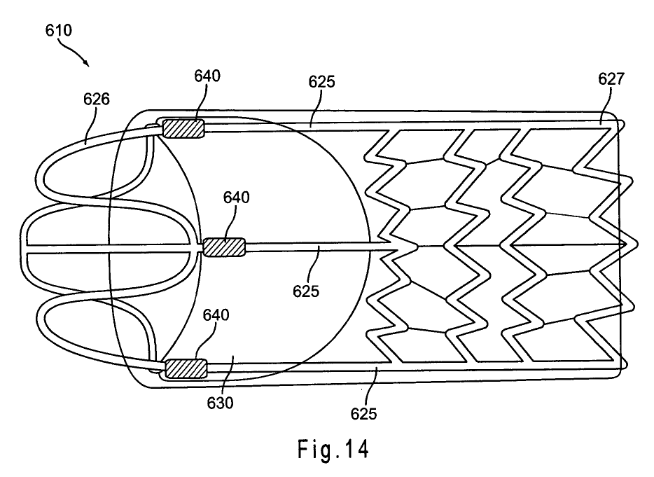

FIG. 14 illustrates a medical device 610 according to another embodiment of the present invention. In this embodiment, a frame 620 includes a proximal portion 626 having an open end and a distal portion 627 connected to bar members 625 of the frame 620. The proximal portion 626 has an S shaped configuration and the distal portion 627 has a Z shaped configuration, however, the configuration should not be construed as limiting the scope of invention.

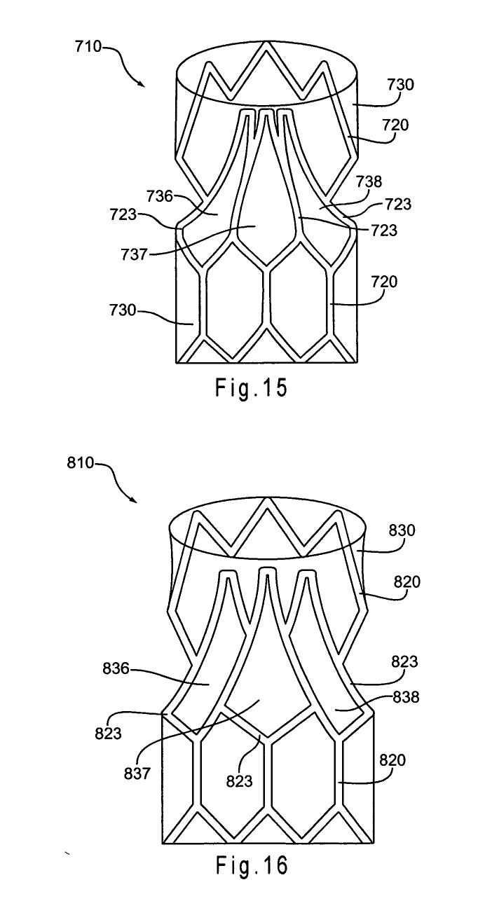

FIGS. 15-16 illustrate a medical device 710 according to another embodiment of the present invention. In this embodiment, the cross-linkable material 730 defines a plurality of leaflets 736, 737, 738 wherein the leaflets are movable between a first and second position when the device 710 is placed in a body lumen. In the first position (see FIG. 14), the leaflets 736, 737, 738 come together to close a valve opening defined by the leaflets 736, 737, 738. In the second position (see FIG. 15), the leaflets 736, 737, 738 open the valve opening which allows blood flow through the valve. By moving between these two positions, the leaflets 736, 737, 738 provide a valving function to the medical device 710, allowing it to regulate fluid flow. The structure of the frame 720 defines a leaf frame 723 corresponding to the shape of the leaflets 736, 737, 738. The leaflets 736, 737, 738 can be attached to leaf frame 723 via cross-linking. For example, each leaflet can be cross-linked to the leaf frame of the device 710, while additional cross-linkable material 730 can be cross-linked to other portions of the frame 720, such as a distal portion or proximal portion of the frame.

Crosslinking

Cross-link bonds can be formed in any suitable manner that provides attachment of a material to an implantable frame, including formation of cross-link chemical bonds between two regions of the material and/or formation of a cross-link bond between the frame and a portion of material. For example, cross-linking can be introduced by chemical treatment of the frame and/or material, such as glycosylation. The material can be subjected to a form of energy to introduce cross-linking. For example, energy treatment suitable for use in the invention includes exposing the material to ultraviolet light, heat, or both. Dehydrothermal treatment (DHT) is one example of a physical method of crosslinking material such as an extracellular matrix material. DHT can be performed by slowly heating the material to a suitable temperature at a reduced pressure. For instance, collagen fibers can be cross-linked by heating to a temperature of about 110° C. at a pressure of about 0.1 μm Hg for a suitable period of time, which can be on the order of up to about 5 days. Alternatively, extracellular matrix material such as collagen can be cross-linked by irradiation at a suitable ultraviolet wavelength, such as 254 nm. For example, irradiation of collagen at 254 nm for about 15-240 minutes at a distance of about 6 inches from a UV light source. DHT and UV irradiation cross linking methods for collagen materials are further compared and discussed in Weadock, et al., “Physical crosslinking of collagen fibers: Comparison of ultraviolet irradiation and dehydrothermal treatment,” H. Biomed. Mat. Res., 29:1373-1379 (1995).

In general, the material for use in the medical device and material for leaflet formation can be processed prior to cross-linking the material. For example, the material can undergo cutting and trimming, sterilizing, and associating the material with one or more desirable compositions, such as anticalcification agents and growth factors, and the like. After any preliminary processing and or storage is completed, the material can be cross-linked. Following cross-linking of the material, the material can be further processed, which can involve additional chemical and or mechanical manipulation of the material as well as processing the material into the desired medical device.

Cross-linking can be performed, for example, to mechanically stabilize the material to the device. Cross-linked material generally refers to material that is completely cross-linked in the sense that further contact with a cross-linking agent does not further change measurable mechanical properties of the material. However, total (100%) cross-linking is not always needed to achieve many desired mechanical properties. Cross-linking of the material preferably involves a chemical cross-linking agent with a plurality of functional groups that bond to the material to form a chemically cross-linked material. The chemical cross-linking is preferably performed until a cross-linking agent has permeated the material of the cross-linking region and reacted with the accessible binding sites of the material.

Once a desired level of cross-linking is reached, the mechanical properties of the material are generally determined or set. While the mechanical properties of cross-linked material are stabilized, the material may gradually change upon exposure to physiological conditions or under inappropriate storage conditions, such as dehydrating conditions for reasonable periods of time. With proper storage, the cross-linked material typically possesses suitably stable mechanical properties. Since the cross-linked material has stable mechanical properties, the mechanical and physical properties of the material can be matched, such that the properties of the respective material is within desired tolerances.

Cross-linking of natural polymers or synthetic polymers can be accomplished with lyophilization, adhesives, pressure and or/heat. Chemical cross-linking can also be used to join layers of material together. Other cross-linking agents can incorporate glutaraldehyde, albumin, formaldehyde or a combination thereof. Material can also be fixed by cross-linking. Fixation provides mechanical stabilization, for example, by preventing enzymatic degradation of the tissue and by anchoring the collagen fibrils.

Other cross-linking agents can be used to form cross-linking regions, such as epoxides, epoxyamines, diimides and other difunctional polyfunctional aldehydes. In particular, aldehyde functional groups are highly reactive with amine groups in proteins, such as collagen. Epoxyamines are molecules that generally include both an amine moiety (e.g. a primary, secondary, tertiary, or quaternary amine) and an epoxide moiety. The epoxyamine compound can be a monoepoxyamine compound and or a polyepoxyamine compound. In some embodiments, the epoxyamine compound is a polyepoxyamine compound having at least two epoxide moieties and possibly three or more epoxide moieties. In some embodiments, the polyepoxyamine compound is triglycidylamine (TGA). The use of cross-linking agents forms corresponding adducts, such as glutaraldehyde adducts and epoxyamine adducts, of the cross-linking agent with the material that have an identifiable chemical structures. Particularly preferred cross-linking agents can be selected from agents useful in cross-linking collagen, such as: diisocyanate compounds (such as hexamethylene diisocyanate), water-soluble carbodiimide (including 1-ethyl-3-(3-dimethyl aminopropyl)carbodiimide, or EDC), N-hydroxysuccinimide (NHS), a combination of EDC and NHS (also called EN-crosslinking), 1,4-butanediol diglycidyl ether (BD), acyl azide and dimethyl suberimidate (DMS). EN-crosslinking can be achieved by contacting the extracellular matrix material with a buffered solution (e.g., pH 5.5) of EDC (e.g., about 1.15 g in 100 mL) and NHS (e.g., about 0.14 g NHS in the same 100 mL) for a time period suitable to achieve crosslinking (e.g., up to about 24 hrs at 20° C.). Alternatively, the material may be crosslinked by contacting a solution of Na₂B₄O₇.10H₂O (e.g., 0.025M at pH 9.0 or 4.5) with BD (e.g., 4 g or 4 wt %) and permitting crosslinking for a desired period of time (e.g., about 144 hours). Crosslinking of collagen tissue with BD, GA and EN is described in van Wachem et al., “In Vivo Behavior of Epoxy-Crosslinked Porcine Heart Valve Cusps and Walls,” J. Biomed. Mater. Res. (Appl. Biomater.) 53:18-27 (2000), the “materials and methods” section of which is incorporated herein by reference. Propyleneimine octaamine dendrimers may also be used as crosslinking agents with extracellular matrix material. For example, diamine(ethylene diamine), triamine (tris-2-aminoethylamine) and dendrimer-crosslinked collagens may be prepared by contacting the material with an aqueous solution of EDC and the multifunctional amine crosslinking agent and a suitablem amount of NHS. The pH of the solution can be adjusted to a desired level, such as 5.5, and crosslinking can be achieved at a suitable temperature, such as about 37° C. Preparation of dendrimer-crosslinked collagens are described in Duan et al., “Crosslinking of collagen with dendrimers,” J. Biomed. Mater. Res. 75A: 510-518 (2005), the “Materials and Methods” section of which is incorporated herein by reference. The preparation of crosslinked extracellular material with DMS can be used to attach the material to a frame containing suitable nitrogen-containing reactive groups, such as amine or amidine groups. For example, collagen material may be crosslinked by contacting the material with an aqueous solution of DMS (e.g., 0.2M) at a suitable pH (e.g., about 9.0) at room temperature for a suitable period of time (e.g., 0.5, 1.0, 1.5, 2.0 and higher). Charulatha et al., “Crosslinking density and resorption of dimethyl suberimidate-treated collagen,” J. Biomed. Mater. Res., 36: 478-486 (1997), the materials and methods section of which is incorporated herein by reference, described methods of crosslinking collagen with a DMS solution.

In general, the process to form completely cross-linked material is conducted for a suitable amount of time. For example, the cross-linking agent may be allowed to penetrate through the material. Also, the cross-linking process generally reaches a point of completion at which time the properties of the material are essentially stable with respect to any additional measurable changes upon further contact with the cross-linking agent. At the point of completion, it is thought that the cross-linking composition forms a stable cross-linked region. Upon completion, the cross-linking is often irreversible such that contact over significant periods of time with aqueous solutions without the cross-linking agent present does not result in reversal of the cross-linking and disassembly of the cross-linked region. Presumably, at completion, many, if not all, of the available functional groups of the material for cross-linking have reacted with a cross-linking agent. Since the formation of a fully cross-linked material is a slow process, the degree of cross-linking of the material at the cross-linking region can be selected to range from very low levels to completion of cross-linking.

Any cross-links can be made via the formation of a chemical bond such as by the reaction of a donor functional group and acceptor functional group. Acceptor functional groups include aldehydes, epoxides, anhydrides, esters, amides, carbonates, urethanes, acetals, ketals, α,β-unsaturated carbonyl compounds and the like. Donor functional groups contain oxygen, nitrogen and sulfur atoms such as alcohols, amines, sulfides, amides and the like. Materials containing nitrogen can be cross-linked with a suitable cross-linking agent containing acceptor functionality such as formaldehyde, glutaraldehyde, polyfunctional aldehydes and epoxides to form a nitrogen-carbon bond such as an aminal or alkylated amine. Glutaraldehyde and polyepoxides are particularly well suited cross-linking agents for nitrogen containing materials because they are difunctional and contain multiple aldehyde and or epoxide functionalities. Other materials containing acceptor functionality such as polyesters, polycarbonates, polyimides, polyurethanes can be cross-linked with a suitable cross-linking agent containing donor functionality such as diamines, polyamines, diols and polyols to form ester, carbonate, amide, urea, and imide bonds. Methods of forming such bonds are well known to one skilled in the art.

In a preferred embodiment, a polyurethane urea, such as the polyureaurethane material sold under the tradename Thoralon®, is cross-linked with small intestine submucosa (SIS). Cross linking of these two materials can be accomplished by reacting the ester functionality of SIS with a cross-linking agent containing an oxygen or nitrogen to form an ester or amide bond, respectively. Polyurethane ureas can be cross-linked by reaction of the urea functionality with an oxygen or nitrogen functionality to form a urea bond or urethane bond. Polyamines, polyalcohols or amino alcohols are suitable cross-linking agent to cross-link polyurethane ureas and SIS. Alternatively, an epoxy amine or epoxy alcohol could be used to cross-link Thoralon and SIS. In this case the amine or alcohol functionality of the cross-linking agent would form an ester or amide bond with the SIS material, and the epoxy functionality of the cross-linking agent would alkylate the urea functionality of the Thoralon.

A remodelable material, such as an extracellular matrix material, may be attached to a frame by first applying a polyurethane coating to the frame, and then cross-linking the remodelable material to the polyurethane coating. A polyurethane frame coating may be applied in any suitable manner, including dipping the frame in a solution comprising the polyurethane, or by spray coating the polyurethane onto the frame. Alternatively, the frame may be formed from the polyurethane material.

The polyurethane material preferably comprises a biocompatible polyurethane material described in U.S. Pat. Application Publication No. 2002/0065552 A1 and U.S. Pat. No. 4,675,361, the disclosures of both of which pertaining to the preparation of polyurethane materials are incorporated herein by reference. The biocompatible polyurethane material sold under the tradename THORALON is a polyurethane base polymer (referred to as BPS-215) blended with a siloxane containing surface modifying additive (referred to as SMA-300). The concentration of the surface modifying additive may be in the range of 0.5% to 5% by weight of the base polymer. One particularly preferred polyurethane material is sold as THORALON (THORATEC, Pleasanton, Calif.). The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference. The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

A variety of other biocompatible polyurethanes/polycarbamates and urea linkages (hereinafter “—C(O)N or CON-type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole. Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. The diamine used as a component of the hard segment includes aliphatic amines, aromatic amines and amines containing both aliphatic and aromatic moieties.

Alternatively, two materials may be cross-linked using radical reactions. A radical is generated in the material to be cross-linked using a free radical generator, such as an organic peroxide of which many are known and commercially available, such as dicumyl peroxide, benzoyl peroxide, and the like. In this case the cross-linking agent is a multifunctional monomer capable of cross-linking the particular polymer when initiated by the free radical generator or irradiation. Typically, the cross-linking agent contains at least two ethylenic double bonds, which may be present, for example, in allyl, methallyl, propargyl or vinyl groups.

In general, material that corresponds to each leaflet can be separately cross-linked. To introduce a desired shape to the material, a segment of material generally is cross-linked in contact with a curved surface for at least a portion of the cross-linking period. The material is contacted with a cross-linking agent for a sufficient period of time to completely crosslink the material. The cross-linking agent can be delivered through one or more apparatuses to perform the cross-linking. The material can be, for example, immersed in the cross-linking agent during the cross-linking process and or cross-linked by spraying the cross-linking agent onto the region of material. For embodiments in which the material is immersed in a cross-linking agent with a self-polymerizing cross-linking agent, the material can be separated from the supply of cross-linking agent by a semi-permeable membrane such that higher molecular weight oligomers polymers of the cross-linking agent are blocked by the semi-permeable membrane.

Materials

A wide variety of materials can be incorporated into the medical device, including polymers, polyesters, and extracellular matrix proteins, such as collagen, gelatin, elastin, glycoproteins, silk collagen/elastic and combinations thereof. Material preferably comprises a cross-linkable polymer. Material is preferably flexible, such as a valve leaflet or portion of biological tissue valve. Material is preferably capable of forming cross link bonds.

The material is also preferably biocompatible, or able to be made biocompatible, and be capable of being cross-linked. Examples of suitable materials include naturally derived materials, and synthetic materials. Examples of suitable natural materials include collagen and extracellular matrix (ECM) material, such as submucosa. Small intestine submucosa (SIS) is particularly well-suited as a material, such as to form valve leaflets. Examples of suitable synthetic materials include polymeric materials, such as polypropylene, polyurethane, expanded polytetrafluoroethylene (ePTFE), polyurethane (PU), polyethylene terphthalate (PET), silicone, latex, polyethylene, polypropylene, polycarbonate, nylon, polytetrafluoroethylene, polyimide, polyester, and mixture thereof, or other suitable materials.

In one embodiment, the material can comprise a substantially biocompatible material, such as polyester fabrics, polytetrafluoroethylene (PTFE), expanded PTFE, and other synthetic materials known to those of skill in the art. In one embodiment, the material can comprise a naturally occurring biomaterial, such as collagen. In one embodiment, the material comprises a collagen material known as extracellular matrix (ECM). In one embodiment, a suitable ECM is small intestinal submucosa (SIS). Other examples of ECMs are pericardium, stomach submucosa, liver basement membrane, urinary bladder submucosa, tissue mucosa, and dura mater.

In one embodiment, the material can comprise a remodelable material. The terms “remodelable” or “bioremodelable” refer to the ability of a material to allow or induce host tissue growth, proliferation or regeneration following implantation of the tissue in vivo. Remodeling can occur in various microenvironments within a body, including without limitation soft tissue, a sphincter muscle region, body wall, tendon, ligament, bone and cardiovascular tissues. Upon implantation of a remodelable material, cellular infiltration and neovascularization are typically observed over a period of about five days to about six months or longer, as the remodelable material acts as a matrix for the ingrowth of adjacent tissue with site-specific structural and functional properties. The remodeling phenomenon which occurs in mammals following implantation of submucosal tissue includes rapid neovascularization and early mononuclear cell accumulation. Mesenchymal and epithelial cell proliferation and differentiation are typically observed by one week after in vivo implantation and extensive deposition of new extracellular matrix occurs almost immediately. In some embodiments, fluid contacting autologous cells on an implanted remodelable material can affect the growth of autologous tissue on the implanted remodelable material.

One example of a suitable remodelable material useful as a material is extracellular matrix (ECM) material derived from submocosal tissue. Submucosal tissue can be obtained from various tissue sources, harvested from animals raised for meat production, including, for example, pigs, cattle and sheep or other mammals. More particularly, the submucosa is isolated from warm-blooded tissues including the alimentary, respiratory, intestinal, urinary or genital tracts of warm-blooded vertebrates.

One preferred category of ECM material is submucosal tissue. Submucosal ECM material can be obtained from any suitable source, including without limitation, intestinal submucosa, stomach submucosa, urinary bladder submucosa, and uterine submucosa. Intestinal submucosal tissue is one preferred starting material, and more particularly intestinal submucosa delaminated from both the tunica muscularis and at least the tunica mucosa of warm-blooded vertebrate intestine. More preferably, the ECM material is Tela submucosa, which is a layer of collagen-containing connective tissue occurring under the mucosa in most parts of the alimentary, respiratory, urinary and genital tracts of animals. Examples of suitable ECM materials include renal capsule matrix (RCM), urinary bladder matrix (UBM) and most preferably small intestine submucosa (SIS). Most preferably, the ECM material is obtained from processed intestinal collagen layer derived from the tunic submucosa of porcine small intestine.

“Tela submucosa” refers to a layer of collagen-containing connective tissue occurring under the mucosa in most parts of the alimentary, respiratory, urinary, integumentary, and genital tracts of animals. Tela submucosa, as with many animal tissues, is generally aseptic in its natural state, provided the human or animal does not have an infection or disease. This is particularly the case since the tela submucosa is an internal layer within the alimentary, respiratory, urinary and genital tracts of animals. Accordingly, it is generally not exposed to bacteria and other cellular debris such as the epithelium of the intestinal tract. Preferably, the tela submucosa tissue ECM materials, which are collagen-based and thus predominantly collagen, are derived from the alimentary tract of mammals and most preferably from the intestinal tract of pigs. A most preferred source of whole small intestine is harvested from mature adult pigs weighing greater than about 450 pounds. Intestines harvested from healthy, nondiseased animals will contain blood vessels and blood supply within the intestinal tract, as well as various microbes such as E. coli contained within the lumen of the intestines. Therefore, disinfecting the whole intestine prior to delamination of the tela submucosa substantially removes these contaminants and provides a preferred implantable tela submucosa tissue which is substantially free of blood and blood components as well as any other microbial organisms, pyrogens or other pathogens that may be present. In effect, this procedure is believed to substantially preserve the inherent aseptic state of the tela submucosa, although it should be understood that it is not intended that the present invention be limited by any theory.

Additional information as to submucosa materials useful as ECM materials herein can be found in U.S. Pat. Nos. 4,902,508; 5,554,389; 5,993,844; 6,206,931; 6,099,567; and 6,375,989, as well as published U.S. Patent Applications US2004/0180042A1 and US2004/0137042A1, which are all incorporated herein by reference. For example, the mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO 98/25636, based on PCT application PCT/US97/23010; or from urinary bladder mucosa as described in U.S. Pat. No. 5,554,389; the disclosures of all are expressly incorporated herein.

A material can comprise an ECM material isolated from biological tissue by a variety of methods. In general, an ECM material can be obtained from a segment of intestine that is first subjected to abrasion using a longitudinal wiping motion to remove both the outer layers (particularly the tunica serosa and the tunica muscularis) and the inner layers (the luminal portions of the tunica mucosa). Typically the SIS is rinsed with saline and optionally stored in a hydrated or dehydrated state until use as described below. The resulting submucosa tissue typically has a thickness of about 100-200 micrometers, and may consist primarily (greater than 98%) of acellular, eosinophilic staining (H&E stain) ECM material.

Preferably, the source tissue for the ECM material is a tela submucosa that is disinfected prior to delamination by the preparation disclosed in US Patent Application US2004/0180042A1 by Cook et al., published Sep. 16, 2004 and incorporated herein by reference in its entirety. Most preferably, the tunica submucosa of porcine small intestine is processed in this manner to obtain the ECM material. This method is believed to substantially preserve the aseptic state of the tela submucosa layer, particularly if the delamination process occurs under sterile conditions. Specifically, disinfecting the tela submucosa source, followed by removal of a purified matrix including the tela submucosa, e.g. by delaminating the tela submucosa from the tunica muscularis and the tunica mucosa, minimizes the exposure of the tela submucosa to bacteria and other contaminants. In turn, this enables minimizing exposure of the isolated tela submucosa matrix to disinfectants or sterilants if desired, thus substantially preserving the inherent biochemistry of the tela submucosa and many of the tela submucosa's beneficial effects.

Preferably, the ECM material is substantially free of any antibiotics, antiviral agents or any antimicrobial type agents which may affect the inherent biochemistry of the matrix and its efficacy upon implantation. An alternative to the preferred method of ECM material isolation comprises rinsing the delaminated biological tissue in saline and soaking it in an antimicrobial agent, for example as disclosed in U.S. Pat. No. 4,956,178. While such techniques can optionally be practiced to isolate ECM material from submucosa, preferred processes avoid the use of antimicrobial agents and the like which may not only affect the biochemistry of the collagen matrix but also can be unnecessarily introduced into the tissues of the patient.

Other disclosures of methods for the isolation of ECM materials include the preparation of intestinal submucosa described in U.S. Pat. No. 4,902,508, the disclosure of which is incorporated herein by reference. Urinary bladder submucosa and its preparation is described in U.S. Pat. No. 5,554,389, the disclosure of which is incorporated herein by reference. Stomach submucosa has also been obtained and characterized using similar tissue processing techniques, for example as described in U.S. patent application Ser. No. 60/032,683 titled STOMACH SUBMUCOSA DERIVED TISSUE GRAFT, filed on Dec. 10, 1996, which is also incorporated herein by reference in its entirety.

In some embodiments, submucosal tissues for use in accordance with this invention include intestinal submucosa, stomach submucosa, urinary bladder submucosa, and uterine submucosa. Another specific example of a suitable remodelable material is intestinal submucosal tissue, and more particularly intestinal submucosa delaminated from both the tunica muscularis and at least the tunica mucosa of warm-blooded vertebrate intestine.

SIS can be made, for example, in the fashion described in U.S. Pat. No. 4,902,508 to Badylak et al., U.S. Pat. No. 5,733,337 to Carr, and WIPO Patent No. WO 9822158, published May 28, 1998, issued to Cook Biotech Inc. et al. and listing Patel et al. as inventors. The preparation and use of SIS is also described in U.S. Pat. Nos. 5,281,422 and 5,275,826. Urinary bladder submucosa and its preparation is described in U.S. Pat. No. 5,554,389, the disclosure of which is expressly incorporated herein by reference. The use of submucosal tissue in sheet form and fluidized forms for inducing the formation of endogenous tissues is described and claimed in U.S. Pat. Nos. 5,281,422 and 5,275,826, the disclosures of which are expressly incorporated herein by reference.

A variety of remodelable materials are known in the art, including naturally derived or synthetic collagenous materials that are capable of providing remodelable surfaces on implantable medical devices. Examples of material materials useful in certain embodiments of the present invention also include: the regenerative compositions comprising epithelial basement membranes as described in U.S. Patent No. 6,579,538 to Spievack, the remodelable implantable valves described in U.S. Pat. No. 6,126,686 to Badylak et al., the enzymatically digested submucosal gel matrix composition of U.S. Pat. No. 6,444,229 to Voytik-Harbin et al., materials comprising the carboxy-terminated polyester ionomers described in U.S. Pat. No. 5,668,288 to Storey et al., the biodegradable surgical implant of U.S. Pat. No. 6,171,338 to Talja et al., collagen-based matrix structure described in U.S. Pate. No. 6,334,872 to Termin et al., the autologous tissue venous valve described in U.S. Pat. No. 6,562,068 to Drasler et al., and combinations thereof.

Although certain embodiments of the present invention provide a material comprising remodelable material, the invention is not limited to material materials that are remodelable. Any material known in the art that provides a desirable property that is responsive to fluid contacting the surface when implanted in vivo can be used to provide a material.

Any suitable implantable material or portion of an implantable material, including those described above, can serve as the material. The examples recited above provide illustrative examples of some suitable materials that can be selected to provide a material. Other materials known in the art can also be selected to provide a material for some embodiments of the present invention.

Frames

Type of cross linking also depends on the type of frame. In one embodiment, the frame is a metal and a point of attachment is formed by joining multiple portions of the material via cross-linking modification of the material. In another embodiment, the frame cross-linked to the material.

Any suitable support frame can be used as the support frame in the medical device. The specific support frame chosen will depend on several considerations, including the size and configuration of the vessel and the size and nature of the medical device.

A support frame that provides a stenting function, i.e., exerts a radially outward force on the interior of the body vessel in which the medical device is implanted, can be used if desired. By including a support frame that provides a stenting function, the medical device can provide a stenting functionality at a point of treatment in a body vessel. The stent art provides numerous examples of support frames acceptable for use in the medical device, and any suitable stent can be used as the support frame. If a stent is used, the specific stent chosen will depend on several factors, including the vessel in which the medical device is being implanted, the axial length of the treatment site, the number of valves desired in the device, the inner diameter of the body vessel, the delivery method for placing the support structure, and others. Those skilled in the art can determine an appropriate stent based on these and other factors.

The support frame can have any suitable size. The exact configuration and size chosen will depend on several factors, including the desired delivery technique, the nature of the body vessel in which the medical device will be implanted, and the size of the vessel. The support frame can be sized so that the second, expanded configuration is slightly larger in diameter that the inner diameter of the vessel in which the medical device will be implanted. This sizing can facilitate anchoring of the medical device within the body vessel and maintenance of the medical device at a point of treatment following implantation.

Suitable support frames can also have a variety of configurations, including braided strands, helically wound strands, ring members, consecutively attached ring members, tube members, and frames cut from solid tubes. Also, suitable frames can have a variety of sizes. The exact configuration and size chosen will depend on several factors, including the desired delivery technique, the nature of the vessel in which the device will be implanted, and the size of the vessel. A frame structure and configuration can be chosen to facilitate maintenance of the device in the vessel following implantation.

The support frame can be made from one or more suitable materials. Examples of suitable materials include, without limitation: stainless steel (such as 316 stainless steel), nickel titanium (NiTi) alloys (such as Nitinol) and other shape memory and/or superelastic materials, MP35N, gold, silver, a cobalt-chromium alloy, tantalum, platinum or platinum iridium, or other biocompatible metals and/or alloys such as carbon or carbon fiber, cellulose acetate, cellulose nitrate, silicone, cross-linked polyvinyl alcohol (PVA) hydrogel, cross-linked PVA hydrogel foam, polyurethane, polyamide, styrene isobutylene-styrene block copolymer (Kraton), polyethylene teraphthalate, polyurethane, polyamide, polyester, polyorthoester, polyanhidride, polyether sulfone, polycarbonate, polypropylene, high molecular weight polyethylene, polytetrafluoroethylene, or other biocompatible polymeric material, or mixture of copolymers thereof, or stainless steel, polymers, and any suitable composite material.

In one embodiment, the frame is self-expanding. Upon compression, self-expanding frames can expand toward their pre-compression geometry. In some embodiments, a self-expanding frame can be compressed into a low-profile delivery conformation and then constrained within a delivery system for delivery to a point of treatment in the lumen of a body vessel. At the point of treatment, the self-expanding frame can be released and allowed to subsequently expand to another configuration. In certain embodiments, the frame is formed partially or completely of alloys such as nitinol (NiTi) which have superelastic (SE) characteristics. However, while some embodiments provide frames made from shape memory materials, other embodiments comprise other materials such as stainless steel, MP35N and other suitable materials. Some embodiments provide frames that are not self-expanding, or that do not comprise superelastic materials.

The support frame can be formed in any suitable shape, including a ring, a stent, a tube, or a zig-zag configuration. In one embodiment, the support frame can be self-expanding or balloon-expandable.

The support frame can be formed from a variety of medical grade polymers having properties that permit the frame to function as a supporting structure for the remodelable material. In some embodiments, the support frame comprises a bioabsorbable or remodelable material.

The support frame can comprise a bioabsorbable material that can be degraded and absorbed by the body over time to advantageously eliminate a frame structure from the vessel before, during or after the remodeling process. A number of bioabsorbable homopolymers, copolymers, or blends of bioabsorbable polymers are known in the medical arts. These include, but are not necessarily limited to, polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-alpha-hydroxy acids, trimethlyene carbonate, poly-beta-hydroxy acids, polyorganophosphazines, polyanhydrides, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, modified polysaccharides and modified proteins.

Some specific examples of bioabsorbable materials include polymers and co-polymers comprising a polylactic acid, a polyglycolic acid, a polycaprolactone or derivatives thereof. Suitable bioabsorbable materials for a frame include: poly(epsilon-caprolactone), poly(dimethyl glycolic acid), poly(hydroxy butyrate), poly(p-dioxanone), polydioxanone, PEO/PLA, PLA, poly(lactide-co-glycolide), poly(hydroxybutyrate-co-valerate), poly(glycolic acid-co-trimethylene carbonate), poly(epsilon-caprolactone-co-p-dioxanone), poly-L-glutamic acid or poly-L-lysine, polylactic acid, polylactide, polyglycolic acid, polyglycolide, poly(D,L-lactic acid), L-polylactic acid, poly(glycolic acid), polyhydroxyvalerate, cellulose, chitin, dextran, fibrin, casein, fibrinogen, starch, collagen, hyaluronic acid, hydroxyethyl starch, and gelatin. A frame may also comprise one or more naturally derived bioabsorbable polymers, including modified polysaccharides such as cellulose, chitin, and dextran or modified proteins such as fibrin and casein.

The frame can include structural features, such as barbs, that maintain the frame in position following implantation in a body vessel. The art provides a wide variety of structural features that are acceptable for use in the medical device, and any suitable structural feature can be used. Furthermore, barbs can also comprise separate members attached to the frame by suitable attachment means, such as welding and bonding. For instance, barbs can be formed by V-shaped cuts transversing the thickness of a flat metal frame, which are bent outward to form the barb. In some embodiments, the number, arrangement, and configuration of the integral barbs can vary according to design preference and the clinical use of the device. The barbs can have any suitable shape, including points or “fish hook”-like configurations. The barbs may or may not penetrate the vein wall, depending on their design and other factors, including the thickness and type of covering used.

Also provided are embodiments wherein the frame comprises a means for orienting the frame within a body lumen. For example, the frame can comprise a marker, such as a radiopaque portion of the frame that would be seen by remote imaging methods including X-ray, ultrasound, Magnetic Resonance Imaging and the like, or by detecting a signal from or corresponding to the marker. In other embodiments, the delivery device can comprise a frame with indicia relating to the orientation of the frame within the body vessel. In other embodiments, indicia can be located, for example, on a portion of a delivery catheter that can be correlated to the location of the frame within a body vessel.

A frame or delivery device may comprise one or more radiopaque materials to facilitate tracking and positioning of the medical device, which may be added in any fabrication method or absorbed into or sprayed onto the surface of part or all of the medical device. The degree of radiopacity contrast can be altered by implant content. Radiopacity may be imparted by covalently binding iodine to the polymer monomeric building blocks of the elements of the implant. Common radiopaque materials include barium sulfate, bismuth subcarbonate, and zirconium dioxide. Other radiopaque elements include: cadmium, tungsten, gold, tantalum, bismuth, platinum, iridium, and rhodium. In one preferred embodiment, iodine may be employed for its radiopacity and antimicrobial properties. Radiopacity is typically determined by fluoroscope or x-ray film. Various other ways to incorporate radiopaque material in a medical device are provided in copending application Ser. No. 10/787,307, filed Feb. 26, 2004 by Case et al., entitled “Prosthesis Adapted for Placement Under External Imaging,” which is incorporated herein by reference. Imagable markers, including radiopaque material, can be incorporated in any portion of a medical device. For example, radiopaque markers can be used to identify a long axis or a short axis of a medical device within a body vessel. For instance, radiopaque material may be attached to a frame or woven into portions of the valve member material.

Delivery

Preferably, the medical device is implanted in a compressed configuration, and expanded at a point of treatment within a body vessel. The overall configuration, cross-sectional area, and length of the frame in the tubular configuration (compressed or expanded) will depend on several factors, including the size and configuration of device, the size and configuration of the vessel in which the device will be implanted, the extent of contact between the device and the walls of the vessel, and the amount of retrograde flow through the vessel that is desired.

Although the medical device is shown with two valve members, other embodiments provide medical devices comprising 1, 3, 4, 5, 6, 7, 8 or more valve members. The valve members can be arranged in any suitable configuration with respect to one another and the frame. In one preferred embodiment, a medical device can comprise a frame and three valve members that are leaflets comprising free edges. In another preferred embodiment, a medical device can comprise one leaflet having a free edge that can sealably engage the interior of a vessel wall. Other suitable configurations of valve members are provided by further embodiments, including differently shaped valve members, and different points of attachment by valve members to the frame.

In one embodiment, the valve members are substantially oriented parallel to the longitudinal axis of a medical device. The orientation of a valve member in a medical device is discussed in U.S. patent application Ser. No. 10/787,307, filed Feb. 26, 2004 (by Case et al.), entitled “Prosthesis Adapted for Placement Under External Imaging,” which is incorporated herein by reference.

In devices including multiple openings that permit a controlled amount of fluid flow in the second, opposite direction to flow through the vessel in which the device is implanted, the total open area of all openings can be optimized as described above, but it is not necessary that the individual openings have equivalent total open areas.

Methods of Manufacturing

In some embodiments, the medical devices can be configured for delivery to a body vessel. For example, a medical device can be compressed to a delivery configuration within a retaining sheath that is part of a delivery system, such as a catheter-based system. Upon delivery, the delivery configuration can be expanded, for example, by removing a self-expanding frame, or portion thereof, from the sheath or by inflating a balloon from inside the medical device. The delivery configuration can be maintained prior to deployment of the medical device by any suitable means, including a sheath, a suture, a tube or other restraining material around all or part of the compressed medical device, or other methods. A method of making an implantable medical device can comprise providing a frame and covering the frame with a cross-linkable material. The method can further comprise cross-linking at least one region of the cross-linkable material, wherein the cross-linking comprises forming a cross-linked region joining a first region of the cross-linkable material with a second region of the cross-linkable material.

In some embodiments, a bioabsorbable suture or sheath can be used to maintain a medical device in a compressed configuration both prior to and after deployment. As the bioabsorbable sheath or suture is degraded by the body after deployment, the medical device can expand within the body vessel. In some embodiments, a portion of the medical device can be restrained with a bioabsorbable material and another portion allowed to expand immediately upon implantation. For example, a self-expanding frame can be partially restrained by a bioabsorbable material upon deployment and later expand as the bioabsorbable material is absorbed.

The invention also provides methods of making medical devices for implantation in a body vessel. In one embodiment, the method comprises the step of attaching a first valve member to a frame. The valve member can be responsive to the flow of fluid through the frame, and adapted to permit fluid flow through said vessel in a first direction or substantially prevent fluid flow through said vessel in a second, opposite direction. The frame can have a longitudinal axis, a first radial compressibility along a first radial direction that is less than a second radial compressibility along a second radial direction.

Methods of Treatment

The invention also provides methods of treating a patient. In one embodiment the method comprises a step of delivering a medical device as described herein to a point of treatment in a body vessel, and deploying the medical device at the point of treatment. Additionally, the medical device can comprise a frame and a cross-linkable material having a cross-linked region where the cross-linked region maintains the material in connection to the frame.

The delivering step can comprise delivery by surgical or by percutaneous delivery techniques known to those skilled in the art.

Still other embodiments provide methods of treating a subject, which can be animal or human, comprising the step of providing one or more frames as described herein. Other methods further comprise the step of providing one or more frames attached to one or more valve members, as described herein. In some embodiments, methods of treating may also provide the step of delivering a medical device to a point of treatment in a body vessel, or deploying a medical device at the point of treatment, wherein the medical devices are as described herein.

Methods for treating certain conditions are also provided, such as venous valve insufficiency, varicose veins, esophageal reflux, restinosis or atherosclerosis. In some embodiments, the invention relates to methods of treating venous valve related conditions.

A “venous valve related condition” is any condition presenting symptoms that can be diagnostically associated with improper function of one or more venous valves. In mammalian veins, natural valves are positioned along the length of the vessel in the form of leaflets disposed annularly along the inside wall of the vein which open to permit blood flow toward the heart and close to prevent back flow. These natural venous valves act as open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuosity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

Methods for delivering a medical device as described herein to any suitable body vessel are also provided, such as a vein, artery, billiary duct, ureteral vessel, body passage or portion of the alimentary canal.

The invention includes other embodiments within the scope of the claims, and variations of all embodiments.

We claim:

1. A medical device for implantation in a body vessel, comprising:
a frame; and
a cross-linkable material having at least one cross-linked region and a non cross-linked region, where the cross-linked region maintains the cross-linkable material in connection to the frame.

2. The medical device of claim 1, wherein the cross-linked region comprises cross-link bonds between a first region and a second region of the cross-linkable material.

3. The medical device of claim 1, wherein the cross-linked region is cross-linked to a first surface of the frame.

4. The medical device of claim 1, wherein the cross-linkable material is moveable relative to the frame.

5. The medical device of claim 1, wherein the cross-linkable material comprises extra cellular matrix material.

6. The medical device of claim 5, wherein the extra cellular matrix material comprises small intestine submucosa.

7. The medical device of claim 1, wherein the cross-linkable material comprises a polymer.

8. The medical device of claim 1, wherein the cross-linkable material comprises a cross-linkable tissue.

9. The medical device of claim 1, wherein the frame comprises a material selected from the group consisting of nickel, titanium, cobalt-chromium and stainless steel.

10. The medical device of claim 1, wherein the cross-linkable material defines a lumen comprising a valve means for regulating fluid flow through the lumen.

11. The medical device of claim 1, wherein the cross-linked region comprises cross-link bonds between a sugar and a protein.

12. The medical device of claim 1, wherein the frame comprises a cross-linkable polymer.

13. The medical device of claim 1, wherein the frame comprises small intestine submucosa.

14. A method of making an implantable medical device comprising:
providing a frame;
covering the frame with a cross-linkable material; and
cross-linking at least one region of the cross-linkable material.

15. The method of claim 14, wherein the cross-linking comprises forming a cross-linked region joining a first region of the cross-linkable material with a second region of the cross-linkable material.

16. The method of claim 15, wherein the cross-linked region is cross-linked to a first surface of the frame.

17. The method of claim 14, further comprising trimming excess cross-linkable material from the implantable medical device.

18. The method of claim 14, wherein the cross-linkable material is cross-linked by immersing the cross-linkable material in a cross-linking agent.

19. The method of claim 14, wherein the cross-linkable material forms cross-links when treated with glutaraldehyde.

20. The method of claim 14, wherein the cross-linkable material forms cross-links when treated with epoxyamine.

21. The method of claim 14, wherein the cross-linkable material is cross-linked by spraying the cross-linking agent onto the cross-linkable material.

22. A method of treating a subject comprising:
implanting a medical device at a point of treatment, the medical device comprising a frame and a cross-linkable material having a cross-linked region where the cross-linked region maintains the cross-linkable material in connection to the frame.
